(19) United States
(12) Patent Application Publication
(10) Pub. No.: US 2026/0338290 A1
(43) Pub. Date: Oct. 29, 2026

(54) WASTE COLLECTION SYSTEM AND BASE CABINET COMPRISING A WASTE COLLECTION SYSTEM

(71) Applicant: BLANCO GMBH + CO KG, Oberderdingen (DE)

(72) Inventors: Christian KOELLING, Bretten (DE); Romina GAENSLER, Kirchardt (DE)

(73) Assignee: BLANCO GMBH + CO KG, Oberderdingen (DE)

(21) Appl. No.: 18/997,345

(22) PCT Filed: Jul. 19, 2023
(86) PCT No.: PCT/DE2023/200151

§ 371 Date: Jan. 21, 2025

(30) Foreign Application Priority Data
Jul. 21, 2022 (DE) 10 2022 207 501.3

(51) Int. Cl. F16K 24/00; A61L 2/202; A61L 9/20; A61L 103/00; B65F 1/14; G05D 7/06

(52) U.S. Cl. CPC F16K 24/00; A61L 2/202; A61L 9/20; B65F 1/1426; G05D 7/0635; A61L 2103/23; A61L 2202/14; B65F 2210/128; B65F 2210/129; B65F 2210/137; B65F 2210/168

(57) ABSTRACT

A waste collection system has a container for receiving waste. The container has an opening and a discharge device for discharging a gas, in particular air, out of the container via the opening of the container. A sensor device has a first sensor for ascertaining a characteristic, in particular the moisture of at least one gas in the container, and a second sensor for ascertaining the characteristic. The second sensor is arranged to detect the characteristic of the gas outside the container. A controller is connected to the sensor device and the discharge device so that the discharge device can be operated by the controller based on sensor values of the first and second sensors.

### CROSS-REFERENCE TO RELATED APPLICATIONS

This application is a national stage application filed under 35 U.S.C. §371 of PCT Application No. PCT/DE2023/200151, filed Jul. 19, 2023, which claims priority to German patent application DE 10 2022 207 501.3, filed Jul. 21, 2022, the entire disclosure of each is incorporated herein by reference.

### BACKGROUND OF THE DISCLOSURE

### 1. Field of the Disclosure

The disclosure relates to a waste collection system comprising at least one container for receiving waste, wherein the at least one container has at least one opening and a discharge device for discharging at least one gas from the at least one container, in particular air, via the at least one opening of the container.

The disclosure also relates to a base cabinet with a waste collection system.

Although applicable to any waste collection system, the present disclosure is described in relation to waste collection systems in kitchen base cabinets.

### 2. Description of Related Art

Waste collection systems for collecting kitchen waste have been around for a long time. It has become known to provide a waste bin in a base cabinet, for example of a sink or the like, which has an opening in the upper area, is covered by a shelf or the like when closed and is arranged so that it can be pulled out, so that the opening of the container is accessible to a user when pulled out.

DE 10 2010 048 221 A1 discloses a waste collector for kitchen waste which prevents odor and mould formation by allowing air to flow through one or more waste collection containers by removing moisture and odorous substances. The exhaust air produced is disposed of via an exhaust air or drain. The design of the system is modular, and the extraction device can be connected directly to the drain. A valve protects against the backflow of odorous air. The shape causes the air in the waste container to form at least one vortex.

The disadvantage of this is that energy-efficient operation of the extraction device is not possible. In addition, the design is expensive and complex and retrofitting into existing waste collection systems is not possible or only possible with considerable redesign.

### SUMMARY OF THE DISCLOSURE

It is therefore a task of the present disclosure to provide a waste collection system and a base cabinet with a waste collection system, which enable energy-efficient operation of the waste collection system. A further object of the present disclosure is to provide a waste collection system and a base cabinet with waste collection system, which are easy and inexpensive to manufacture and operate. A further object of the present disclosure is to provide an alternative waste collection system and an alternative base cabinet with a waste collection system.

In one embodiment, the present disclosure solves the above-mentioned tasks in a waste collection system comprising at least one container for receiving waste, the at least one container having at least one opening, and a discharge device for discharging at least one gas from the at least one container, in particular air, via the at least one opening of the container, in that a sensor device is arranged which comprises at least one first sensor for determining a parameter, in particular the humidity of at least one gas in the container, and at least one second sensor for determining the parameter, which is arranged to detect the parameter of at least one gas outside the container, and in that a control device is arranged, which is connected to the sensor device and discharge device, in such a way that the discharge device can be operated by means of the control device as a function of sensor values of the at least two sensors of the sensor device.

In one embodiment, the present disclosure solves the aforementioned problems with a base cabinet with a waste collection system according to the disclosure.

One of the advantages of this is that two sensors, in particular in the form of humidity sensors, can be used to determine the respective parameter, for example, the humidity inside and outside the container. In this way, it can be determined whether the humidity is generally high in the base cabinet or only within the container. This enables more energy-efficient operation, especially only when the humidity in the container is high and the ambient humidity is low.

The term “parameter” is to be understood in the broadest sense and refers, in particular, in the claims, preferably in the description, to any kind of physical or chemical quantity that can be measured by means of a sensor. In particular, the “parameter” refers to waste in general, preferably kitchen waste in particular. In other words, this refers in particular to parameters that can directly or indirectly describe or measure a state of waste, for example, the composition of the waste, released gases and/or liquids and their composition, moisture and/or gases in the waste container, volume of the waste, residual volume in the container and the like.

Further features, advantages and further embodiments of the disclosure are described below or are disclosed thereby.

According to an advantageous further development of the disclosure, the sensor device has a further sensor for detecting putrefaction gases, which is arranged to detect at least one putrefaction gas in and/or outside the container. One advantage of this is that even harmful gases can be detected at an early stage and removed quickly.

According to another advantageous further development of the disclosure, the discharge device has a controllable discharge element which is arranged in the center of the at least one opening. In this way, a particularly efficient, centralized discharge of gases through the discharge device from the inside of the container is possible. For example, one or more fans, in particular one or more radial fans, can be used as a controllable discharge element.

According to another advantageous further development of the disclosure, the discharge device has a controllable discharge element that is arranged at the side of the at least one opening. This allows a cover plate or similar to be arranged above the opening of the container, for example, to optimize the installation space. Alternatively or additionally, a centralized discharge can also be provided.

According to another advantageous further development of the disclosure, the discharge device has at least one backflow prevention element, in particular in the form of a flap and/or a valve. This is an efficient and simple way of preventing the backflow of unwanted odors. For example, a mechanical and/or electronic non-return valve, a non-return valve with spring and/or ball and/or disc, a non-return valve with a flexible membrane, a membrane, a Tesla valve and/or a duckbill valve, or the like can be used as a backflow prevention element.

According to another advantageous further development of the disclosure, the at least one backflow prevention element is arranged downstream of the controllable discharge element. In this way, energy can be saved, as the controllable discharge element, for example, a fan, if it is arranged at a distance of 15 cm or less, preferably 10 cm or less, in particular 5 cm or less, for example, has to apply less pressure and can therefore be operated at a lower speed and lower noise level.

According to another advantageous further development of the disclosure, a detection device is arranged which is designed to detect an open or closed state of the at least one opening of the container, the discharge device being controllable depending on the detected state of the at least one opening. The advantage of this is the automatic control of the discharge device depending on the opening state of the container, which enables energy-efficient operation. This means that the discharge device can be switched off when the container is open and only switched on again when the container closes or is closed.

According to another advantageous further development of the disclosure, the detection device comprises a button, a switch, in particular a magnetic switch, and/or a detection sensor, in particular in the form of a LIDAR sensor and/or a light sensor, a light barrier, a force sensor, a magnetic field sensor and/or an ultrasonic sensor. This enables cost-effective and/or reliable detection of the opening state of the container.

According to another advantageous further development of the disclosure, the control device has a wireless interface for communicating with a user of the waste collection system. The advantage of this is that a user can, for example, be shown the sensor values received. The sensor values can also be saved and shown on a display, for example, from a smartphone, a display interface on or in the base cabinet, a display projected onto a worktop above the base cabinet, or similar. The wireless interface can be designed as a W-LAN, Bluetooth, infrared, and/or WiFi interface, for example.

According to another advantageous further development of the disclosure, at least one element, in particular the controllable discharge element, is mounted in a sound-decoupled manner. The advantage of this is particularly quiet and vibration-free operation. Decoupling elements such as rubber buffers or similar can be used for this purpose.

According to another advantageous further development of the disclosure, an ozone device is arranged which is designed to introduce ozone gas into the container, in particular wherein an ozone measuring sensor for determining a concentration and/or quantity of ozone gas is arranged inside and/or outside the container. The advantage of this is that putrefaction gases can be efficiently suppressed or at least reduced, which lowers the overall odor nuisance even when the container is open. The ozone device can be arranged inside the container, for example below an intermediate shelf or similar, and/or outside the container, for example in a base cabinet, and with a corresponding duct for pressurising the inside of the container with ozone gas.

According to another advantageous further development of the disclosure, the discharge device is designed as an air recirculation device so that the at least one gas discharged from the at least one opening is treated by means of a treatment device and supplied to an environment of the container. The advantage of this is that there is no need for a costly further discharge of at least one gas. Alternatively or additionally, it is also possible to return the treated gas directly to the container.

According to another advantageous further development of the disclosure, the treatment device has a fresh air supply device, a filter device and/or an irradiation device, in particular by means of UV-C light. The advantage of this is that with a filter device, for example, the discharged air can be filtered and thus the odor load can be reduced, especially when it is returned to the base cabinet. Alternatively or additionally, in particular upstream of the filter device, the at least one gas can be irradiated with UV-C light. This produces smaller quantities of ozone gas and at the same time kills microorganisms, which further reduces odor load. By means of a fresh air supply device, fresh air can be supplied to the treatment unit from outside and mixed with the discharged air in order to further reduce odor load during recirculation.

According to another advantageous further development of the disclosure, the discharge device is connected to a drain in a region downstream of an odor barrier, in particular in the form of a siphon. The advantage of this is that it enables the at least one gas to be simply discharged further, for example outside a building.

Further important features and advantages of the disclosure are apparent from the sub-claims, from the drawings and from the associated descriptions of the figures with reference to the drawings.

It is understood that the above-mentioned features and those to be explained below can be used not only in the combination indicated in each case, but also in other combinations or on their own, without departing from the scope of the present disclosure.

### BRIEF DESCRIPTION OF THE DRAWINGS

Preferred embodiments and embodiments of the present disclosure are shown in the drawings and are explained in more detail in the following description, wherein identical reference signs refer to identical or similar or functionally identical components or elements.

FIG. 1 shows a base cabinet with a waste collection system according to one embodiment of the present disclosure.

FIG. 2 shows a base cabinet with a waste collection system according to one embodiment of the present disclosure.

FIG. 3 shows a base cabinet with a waste collection system according to one embodiment of the present disclosure.

### DETAILED DESCRIPTION OF THE DISCLOSURE

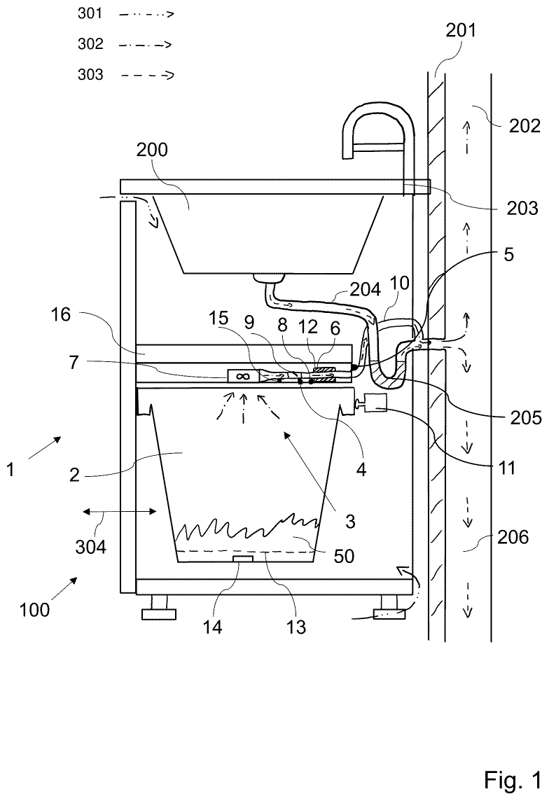

FIG. 1 shows in schematic form a base cabinet with a waste collection system according to one embodiment of the present disclosure.

FIG. 1 shows a base cabinet 100. A basin 200 with a sanitary fitting 203 is arranged in the upper section of the base unit 100. A drain pipe 204 is arranged on the underside of the basin 200, which opens via a siphon 205 into a ventilation pipe 202 for exhaust air 302 or a downpipe 206 for waste water 303 from the basin 200.

A waste collection system 1 is arranged below the basin 200 with a container 2, which can be pulled out horizontally (reference sign 304). The container 2 is open at the top with opening 3. Centrally above the opening 3 and below a horizontal storage shelf 16, which extends essentially over the entire opening 3, a radial fan 7 is arranged, which opens into the drain pipe 204 via a duct 10, in particular partly in the form of a flexible hose, between the siphon 205 and the ventilation line 202. Downstream of the radial fan 7, a non-return flap 9 is arranged in the duct 10 and downstream of the non-return flap 9 a moisture sensor 4 for the container 2, a putrefaction gas sensor 8, for example a VOC sensor, and an ozone gas sensor 15. The above-mentioned sensors can also be arranged outside the duct 10 and arranged together in a housing or separately in separate housings, each with at least one fluid connection to the environment, in particular through a housing bore or the like. A further humidity sensor 5 is also arranged, which can detect the humidity outside the container 2 inside the base cabinet 100.

FIG. 1 shows the container 2 in the non-extended, i.e. closed, state. In this state, the rear area (on the right in FIGS. 1-3) of the container 2 comes into contact with a button 11 in its upper area, with which the closed position of the container 2 shown in FIGS. 1-3 can be detected. If the container 2 is open, the button 11 is not triggered and an open position of the container 2 can be detected in this way. A button 11 is used to detect whether, for example, a base cabinet door with a waste bin is open or closed.

However, the door or waste bin position can also be detected using other sensors, such as infrared sensors, magnetic switches, light detection and ranging sensors (LiDAR sensors), light barriers, force sensors, ultrasonic sensors, photo resistors, magnetic field sensors and/or resistance change sensors. The fan 7 can be switched off, especially when the base cabinet door is opened, so that a user is not disturbed by the fan noise. For example, a noise level of less than 40 dB(A), preferably less than 32 dB(A), can be achieved outside a base cabinet 100 when the waste bin is closed, so that the user is not disturbed.

The container 2 also has a perforated plate 13 in the lower area. An ozone generator 14 is arranged between the perforated plate 13 and the base of the container 2 to generate ozone and feed it into the container 2. Waste 50 in the container 2 collects above the perforated plate 13.

Button 11 and the sensors 4, 5, 8, 15 as well as fan 7 and ozone generator 14 are connected to a control device in the form of a microcontroller 6, which controls the fan 7 and/or the ozone generator 14 accordingly depending on the sensor values of the sensors 4, 5, 8, 15 and button 11, for example, to remove or suppress unwanted odors. The microcontroller 6 can be arranged on a circuit board on the underside of the storage shelf 16. The microcontroller 6, the fan 7, and other electronic components, such as the sensors, can be arranged entirely or at least partially in a housing, which can be made of sheet metal and can be arranged on the underside of the storage shelf 16.

The control device 6 can control the fan 7 as follows: By comparing the two sensor values of the humidity sensors 4, 5, the control device 6 can use the difference between the humidity values inside and outside the container 2 to determine whether there is moist waste 50 in the container 2 and thus operate the fan 7 in particular only when there is moist waste in the container 2. Overall, this allows the fan 7 to be operated shorter and, therefore, more efficiently. The control device 6 can have a wireless interface 12 in order to transmit measured values or the like to a display, a smartphone, a computer, or the like for further evaluation and/or visualization for a user.

Overall, the base cabinet 100 is not hermetically sealed, but supply or fresh air 301 can enter it via gaps between the base cabinet 100 and the worktop or between the house wall 201 and the rear wall of the base cabinet 100.

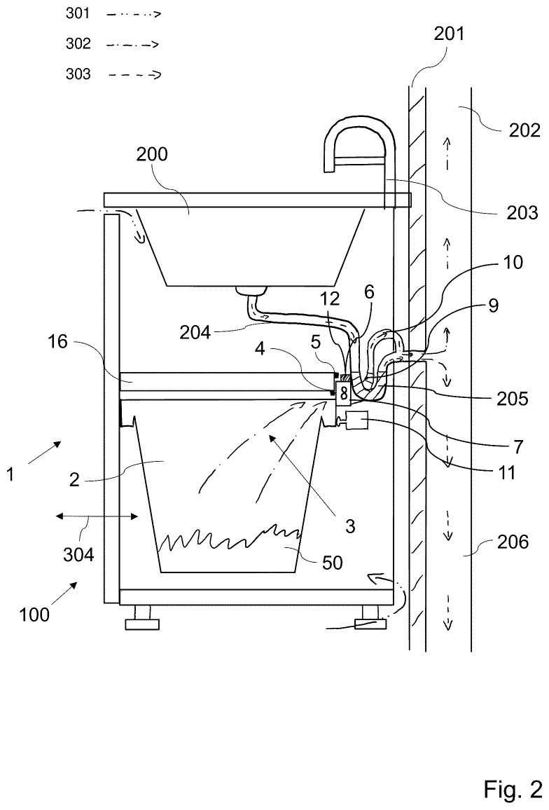

FIG. 2 shows, in schematic form, a base cabinet with a waste collection system according to one embodiment of the present disclosure.

FIG. 2 essentially shows a base cabinet 100 with waste collection system 1 according to FIG. 1. In contrast to the base cabinet 100 with waste collection system 1 according to FIG. 1, the base cabinet 100 with waste collection system 1 according to FIG. 2 does not have an ozone generator 14 or a perforated plate 13. In addition, there is no putrefaction gas sensor 8 and no ozone gas sensor 15. Furthermore, the fan 7 is now arranged in the rear upper area to the side of the opening 3 of the container 2, as is the microcontroller 6.

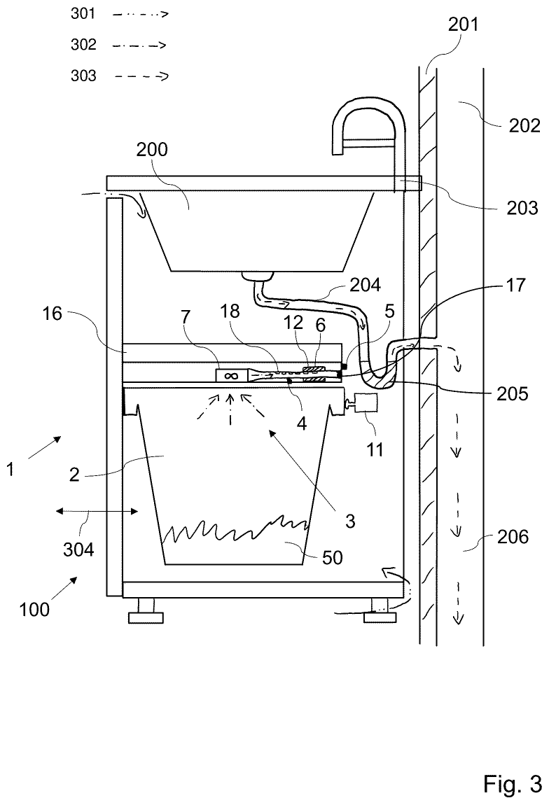

FIG. 3 shows in schematic form a base cabinet with a waste collection system according to one embodiment of the present disclosure.

FIG. 3 essentially shows a base cabinet 100 with waste collection system 1 according to FIG. 1. In contrast to the base cabinet 100 with waste collection system 1 according to FIG. 1, the base cabinet 100 with waste collection system 1 according to FIG. 3 does not have an ozone generator 14 or a perforated plate 13. In addition, there is no putrefaction gas sensor 8 and no ozone gas sensor 15. Furthermore, no duct 10 is arranged to remove exhaust air from the container 2 via a ventilation duct 202. Instead, UV-C LEDs 18 are now arranged downstream of the fan 7 to irradiate the air discharged by the fan 7 with UV-C light, followed by a filter 17 for additional filtering of the air. After irradiation and filtering, the treated air is fed back into the base cabinet 100 outside the container 2.

All embodiments can also be used with several containers 2 and/or with several fans 7. These can then have a common duct for connection behind the siphon 205 or also each have an individual connection hose or line with an individual connection behind the siphon 205. In addition, the fan 7 in particular can be arranged in a sound-decoupled manner from the base cabinet 100. Insulation material can also be installed to reduce airborne noise.

In summary, at least one embodiment of the present disclosure has at least one of the features and/or at least one of the following advantages:
Energy-efficient fan control.
Less pressure loss and less noise.
System communication with a user.
Independent system.
Suitable for many installation situations.
Sound-optimised system.
Reduction of moisture formation in the bin, which reduces mould growth.
Prevents the formation of odors in the room and when the base cabinet is opened, as the extraction system removes bad air and reduces the formation of odors by reducing decomposition.
Flexible usability and retrofittability for existing waste collection systems.

Although the present disclosure has been described with reference to preferred embodiments, it is not limited thereto, but can be modified in a variety of ways.

### LIST OF REFERENCE SYMBOLS

1 Waste collection system
2 Container
3 Opening
4 Humidity sensor
5 Humidity sensor
6 Control device
7 Fan
8 Putrefaction gas sensor
9 Non-return flap
10 Duct
11 Button
12 Wireless interface
13 Perforated plate
14 Ozone generator
15 Ozone gas sensor
16 Storage shelf
17 Filters
18 UV-C LEDS
50 Waste
100 Base cabinet
200 Basin
201 House wall
202 Ventilation pipe
203 Sanitary fitting
204 Drain pipe
205 Siphon
206 Downpipe
301 Supply/fresh air
302 Exhaust air
303 Waste water
304 Pull-out direction/closing direction

1. A waste collection system comprising:
at least one container for receiving waste, the at least one container having at least one opening;
a discharge device for discharging at least one gas from the at least one container via the at least one opening of the container;
a sensor device that comprises at least a first sensor arranged for determining a parameter of at least one gas inside the container and at least a second sensor arranged for determining the parameter of at least one gas outside the container;
a control device connected to the sensor device and the discharge device so that the discharge device can be operated by the control device depending on sensor values of the at least first and second sensors.

2. The waste collection system according to claim 1, wherein the sensor device has a further sensor for detecting putrefaction gases, and wherein the further sensor is arranged to detect at least one putrefaction gas inside and/or outside the container.

3. The waste collection system according to claim 1 wherein the discharge device has a controllable discharge element arranged centrally at the at least one opening.

4. The waste collection system according to claim 1, wherein the discharge device has a controllable discharge element arranged laterally at the at least one opening.

5. The waste collection system according to claim 1, wherein the discharge device has at least one backflow prevention element.

6. The waste collection system according to claim 5, wherein the at least one backflow prevention element is arranged downstream of a controllable discharge element.

7. The waste collection system according to claim 1, further comprising: a detection device that can detect an open or closed state of the at least one opening of the container, wherein the discharge device is controllable depending on a detected state of the at least one opening.

8. The waste collection system according to claim 7, wherein the detection device comprises a button, a switch, a detection sensor, a light barrier, a force sensor, a magnetic field sensor, and/or an ultrasonic sensor.

9. The waste collection system according to claim 1, wherein the control device (6) has a wireless interface for communication with a user of the waste collection system.

10. The waste collection system according to claim 1, wherein the discharge device has a controllable discharge element that is mounted in a sound-decoupled manner.

11. The waste collection system according to claim 1 further comprising: an ozone device that can introduce ozone gas into the container.

12. The waste collection system according to claim 1, wherein the discharge device is configured as an air recirculation device so that the at least one gas discharged from the at least one opening is treated by a treatment device and supplied to an environment of the container.

13. The waste collection system according to claim 12, wherein the treatment device has a fresh air supply device, a filter device, and/or an irradiation device.

14. The waste collection system according to claim 1, wherein the discharge device is connected to a discharge pipe in a region downstream of an odor barrier.

15. A base cabinet comprising a waste collection system according to claim 1.

16. The waste collection system according to claim 1, wherein the at least one gas is air.

17. The waste collection system according to claim 1, wherein the parameter is humidity.

18. The waste collection system according to claim 5, wherein the at least one backflow prevention element is a flap and/or a valve.

19. The waste collection system according to claim 1, further comprising: an ozone device that can introduce ozone gas into the container, and an ozone measuring sensor that can determine a concentration and/or quantity of ozone gas, wherein the ozone measuring sensor is arranged inside and/or outside the container.

20. The waste collection system according to claim 14, wherein the odor barrier is a siphon.
